(19) United States
(12) Patent Application Publication
(10) Pub. No.: US 2011/0046494 A1
(43) Pub. Date: Feb. 24, 2011

(54) Blood Pressure Cuff and Connector Incorporating an Electronic Component

(75) Inventors: Jack Balji, Mahwah, NJ (US); Cadathur Rajagopalan, Dumont, NJ (US)

Correspondence Address: Mindray DS USA, Inc. c/o Stoel Rives LLP, 201 S. Main Street, Suite 1100, Salt Lake City, UT, 84111, US

(73) Assignee: Mindray DS USA, Inc., Mahwah, NJ (US)

(21) Appl. No.: 12/543,673

(22) Filed: Aug. 19, 2009

(51) Int. Cl. A61B 5/022

(52) U.S. Cl. 600/499

(57) ABSTRACT

A blood pressure cuff is equipped with an electronic component providing encoding of cuff properties. The connectors and hose connecting the cuff to a blood pressure measurement instrument are provided with conductors and electrical coupling, allowing the measuring instrument to access the electronic component encoding cuff properties. The arrangement of the cuff, hose, and connectors makes simultaneous pneumatic and electrical connection when the cuff is attached to the hose.

### TECHNICAL FIELD

This disclosure relates generally to non-invasive blood pressure measurement. More specifically, this disclosure relates to a method and device for permitting simultaneous electrical and pneumatic connection to a blood pressure cuff equipped with an electronic component.

### BRIEF DESCRIPTION OF THE DRAWINGS

FIG. 1 depicts the equipment used in the known art of oscillometric blood pressure measurement.

FIG. 2 shows the addition of an electronic component to the blood pressure cuff, and means for connecting the same to a blood pressure monitoring instrument.

FIG. 3 is a schematic diagram showing the connection of the electronic component to the measuring instrument by conductive means.

FIG. 4 shows the connection of the electronic component to the measuring instrument including an electromagnetic coupling.

FIG. 5 shows a combined pneumatic and electrical connector set, in which the electrical connection is adjacent to the pneumatic connection.

FIG. 6 shows a combined pneumatic and electrical connector set, in which the electrical connection is in the form of annular rings, concentric with the pneumatic connection.

FIG. 7 shows a combined concentric pneumatic and electrical connector set, in which the electrical contact is oriented radially.

FIG. 8 shows a sectional view of a male pneumatic coupler containing integral coaxial electrical contacts.

FIG. 9 shows a sectional view of an unmated combined pneumatic and electrical connector set, in which the electrical coupling is by inductive means using adjacent coils.

FIG. 10 shows a sectional view of a mated combined pneumatic and electrical connector set, in which the electrical coupling is by inductive means using coaxial coils.

FIG. 11 shows various forms of passive two-terminal networks which may be used for the cuff electronic component.

FIG. 12 shows the use of capacitor for the electronic component forming a resonant circuit with an inductive coupling coil.

### DETAILED DESCRIPTION

The use of automatic devices for non-invasive blood pressure (NIBP) measurement has become routine in medical practice. Such devices are encountered not only as stand-alone units, but also as integrated functions within multi-parameter medical monitoring devices. Many NIBP devices in common use today operate on the so-called oscillometric principle. In such devices, the only connection to the blood pressure cuff is pneumatic in nature, generally in the form of a hose. This hose is provided in most cases with a connector on each end. One connector allows one end of the hose to be coupled to the NIBP measuring instrument. The second connector allows the blood pressure cuff to be connected to the other end of the hose. In this way, various types of cuffs may be connected to the same hose. Furthermore, in the case of disposable cuffs, the cuff may be replaced without the need to discard the entire hose.

Cuff-based methods of measuring blood pressure rely on inflating and deflating a pneumatic cuff encircling a limb of the body, and noting the pneumatic pressures at which arterial blood flow is completely occluded, corresponding to the systolic blood pressure, and the pneumatic pressure at which no arterial occlusion is produced, corresponding to the diastolic blood pressure. Some methods, such as the auscultatory method, rely on the detection of sounds or vibrations to identify the degree of occlusion, as is commonly done with a stethoscope during manual blood pressure measurements. A salient feature of the oscillometric method is that it allows the blood pressure to be determined solely by observing the pneumatic pressure within the cuff. Minute pulsations, or oscillations, in the cuff pressure are produced when blood flows under the cuff. If the cuff is inflated well above the systolic pressure, the arteries are completely occluded, no blood flows under the cuff, and therefore little or no cuff pressure pulsation is seen. As the cuff pressure is deflated below systolic, blood begins to flow under the cuff during the peak of the blood pressure cycle, and a rapidly increasing cuff pressure pulsation is observed. The amplitude of the cuff pressure pulsation continues to increase until the cuff is deflated past the mean arterial pressure, located part way between systolic and diastolic. The amplitude of the cuff pressure pulsations then begins to decrease as the cuff is further deflated toward the diastolic pressure. In many cases, the decreasing trend somewhat levels off as the cuff is deflated past the diastolic point. By observing the changes in the amplitude of the cuff pressure pulsations relative to the cuff pressure at which they occur, it is possible to identify the systolic, mean, and diastolic blood pressures, using known methods.

Because the oscillometric method operates solely by observation of the cuff pneumatic pressure and pulsations thereof, it may not be necessary to place any sensor or transducer at the patient besides the cuff itself. Further, the connection between the cuff and the measuring instrument may consist only of a pneumatic hose. The cuff pressure and pressure pulsations can be ascertained through the hose by means of transducers or sensors located in the measuring instrument. As such, in commercial oscillometric instruments today the only connection between the patient and the measuring instrument is pneumatic. Some instruments utilize a single hose for the combined purposes of inflating and deflating the cuff, as well as measuring the cuff pressure. However, such a single-hose construction may entail a certain degree of error, due to the pressure drop which results along the length of the hose while air is flowing during cuff deflation. To reduce this source of error, some instruments use a dual hose, or a single hose having two distinct lumens. One hose or lumen is used for airflow necessary to inflate and deflate the cuff, while the other is used solely for pressure measurement. Nevertheless, the connection to the cuff remains purely pneumatic in nature.

Blood pressure cuffs are manufactured in various sizes and types, according to their intended use. Cuffs may be either durable, designed for use on many patients, or designed for disposable application on a single patient. The size of cuffs varies from those intended to fit the thigh of a large adult, to those suitable for the limb of a premature infant. The operation of the NIBP instrument is to some extent influenced by the type and size of cuff connected. This is particularly true for the initial inflation pressure of the cuff. Many instruments will initially inflate an adult cuff to approximately 180 mmHg of pressure, as this is moderately above the presumed normal systolic blood pressure of an adult patient. But such an inflation pressure could prove highly injurious to a neonatal patient, for which a much lower initial pressure is suitable. Many instruments rely on the operator to specify the patient size so that an appropriate initial pressure is used. However, from the standpoint of convenience as well as the safety of small patients, it is preferable that such selection should be entirely automatic. Some instruments attempt to automatically infer the patient size by measuring the size of the attached cuff. Various pneumatic means are used for such determination. For example, the rate of pressure rise when the inflation pump is activated may be used as an indicator of the cuff volume. However, pneumatic means are subject to errors when interfering signal are present, such as when the patient is moving while the cuff size determination is underway.

It may be useful to determine the cuff size for reasons other than selecting the appropriate initial inflation pressure. An NIBP instrument often sets a range of acceptable pulsation amplitudes, with smaller pulses being considered background noise, and larger pulses being considered artifactual. Large cuffs generally develop much larger pulse signals than do small ones. The range of acceptable pulse amplitudes is therefore dependent on the size of the cuff in use, providing another reason why it is desirable to know the cuff size.

Differences in the construction of cuffs can require adjustments to the algorithms employed to determine blood pressure from the pneumatic pulse signal. For example, cuffs designed so as to encircle limbs of the same circumference, but with different width, may produce different blood pressure readings unless corrective measures are taken. Further, cuffs constructed of different materials, such as the different materials used in durable and disposable cuffs, may require similar correction. Therefore, in addition to determining what size patient a cuff is intended for, it may be desirable to obtain information about other cuff characteristics, so that the measuring instrument may suitably adapt, such as by employing a modified pressure determination algorithm or calibration constants.

The pneumatic cuff size determination means found in the known art only attempts to crudely measure cuff volume. Such methods are therefore incapable of discriminating between two cuffs having the same volume, but different shapes. Further, they cannot discriminate between cuffs having other differences, such as material and construction, but nevertheless the same volume. Finally, due to the presence of mechanical interference caused by possible motion of the patient to which the cuff is attached, these methods cannot robustly identify small differences, and may in fact misidentify cuffs altogether.

The hose used to sense the cuff pressure, even in the dual hose system, may introduce artifacts which mask the true cuff pressure or distort or attenuate the pulsations. In these cases, the placement of a pressure or similar sensor on the cuff itself may be useful, but the present system of pneumatic connections does not allow for this.

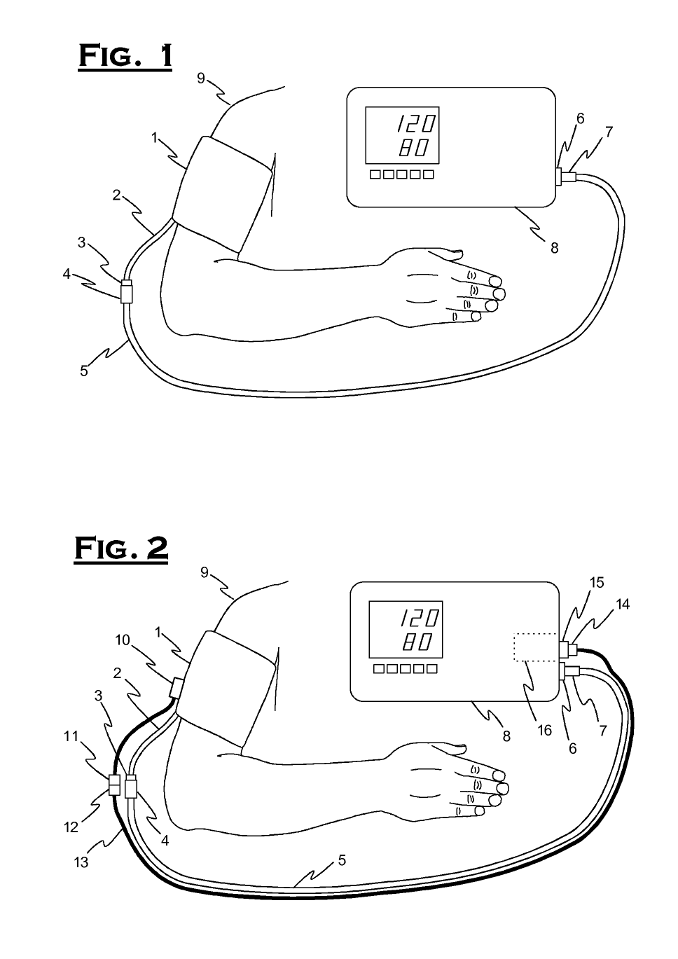

The arrangement of equipment used in the known art of oscillometric blood pressure measurement is illustrated in FIG. 1. A pneumatic blood pressure cuff 1 is wrapped around a limb 9 of a person or animal. The cuff is generally provided with a short tube 2, terminating in pneumatic connector 3. In use, this connector is fitted into connector 4 on the end of hose 5. The other end of the hose is furnished with connector 7, which mates with corresponding pneumatic connector 6 on blood pressure monitoring instrument 8. In some instruments, a dual lumen arrangement is used for hose 5. In this case, the various connector may be duplicated, or may be of such a design as to connect both lumens through independent paths in a single connector body. The shortcomings of this system, with single or dual lumens, are evident in that it permits the exchange of only pneumatic information between the cuff 1 and instrument 8.

This shortcoming is remedied by the instant disclosure, shown in overview in FIG. 2. In addition to the elements found in the known arrangement of FIG. 1, electronic elements have been added. In one embodiment, the cuff 1 is equipped with electronic component 10, which is provided with electrical connector 11. Connector 11 mates with a corresponding electrical connector 12, which is attached to electrical conductors 13 running parallel to hose 5. These conductors terminate at electrical connector 14, which mates with electrical connector 15 on blood pressure monitoring instrument 8. The connector 15 provides access to interface circuit 16 located within instrument 8. Therefore, when all connectors are mated up as shown, the cuff 1 has pneumatic connection to the blood pressure monitoring instrument via hose 5, and the electronic component 10 has electrical connection to interface circuit 16 within instrument 8 via conductors 13.

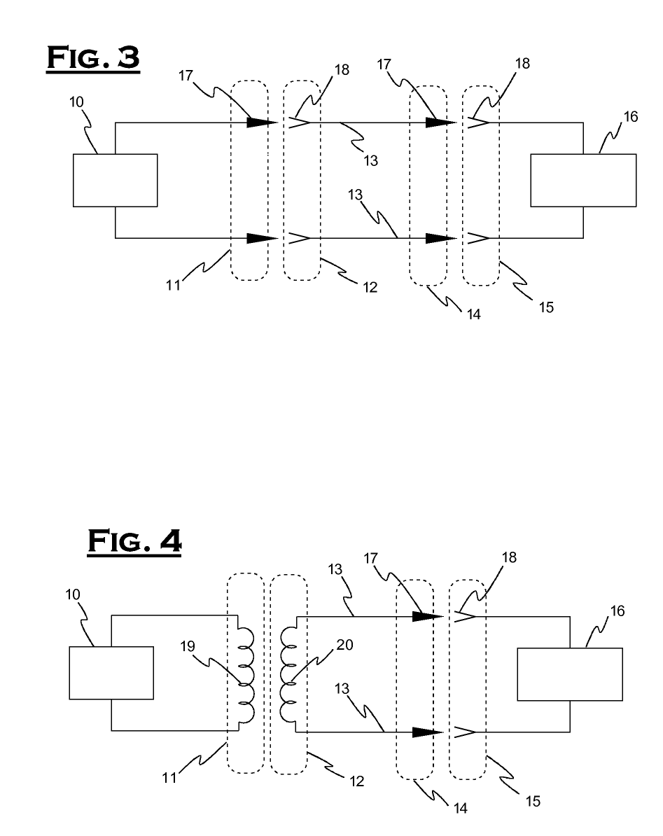

The electrical path between electronic component 10 and interface circuit 16 may take various forms. The most direct form uses conductive connections, one embodiment of which is depicted in FIG. 3. Electronic component 10 is furnished with two or more electrical contacts 17, contained within electrical connector 11. The mating electrical connector 12 is furnished with mating contacts 18, which touch and establish a conductive path to contacts 17 when the connectors are joined. The contacts 18 of connector 12 are attached to conductors 13, which terminate in a similar set of contacts 17 in electrical connector 14, which engage mating contacts 18 in instrument connector 15. The contacts 18 of connector 15 are connected to interface circuit 16. Although the figures show two contacts in each connector, and two conductors, it is understood that more may be provided, according to the requirements of electronic component 10 and interface circuit 16. This arrangement, using a conductive path, is amenable to a wide variety of electronic components, interface circuits, and signaling schemes used to communicate between them.

In an alternate form, at least one of the sets of contacts 17 and mating contacts 18 are replaced by electromagnetic coupling, without touching of contacts. In FIG. 4, the contacts of connector 11 have been replaced by inductive coupling coil 19, and the contacts of connector 12 have been replaced by inductive coupling coil 20. When connector 12 is mated with connector 11, coil 19 is brought into proximity of coil 20, such that the coils become electromagnetically coupled, in the manner of the primary and secondary coils or windings of a transformer. While the figures shows electromagnetic coupling in use at one end only of conductors 13, it is understood that electromagnetic coupling can also be applied in connectors 14 and 15. Hence, it is possible to use electromagnetic coupling in place of contacts at either or both ends of conductors 13.

When coil 19 and coil 20 are brought into proximity, they become coupled as in the case of a transformer. However, according to the construction and arrangement of the coils, the degree of coupling provided may be substantially less than the high degree of coupling commonly provided in transformers. This may be particularly the case when electromagnetic coupling is used at both ends of conductors 13, when the coupling losses become cascaded. However, electronic component 10 and interface circuit 16 may be designed to operate with an arbitrary degree of coupling. The coupling provided by such coils is applicable to AC signals only. Further, coils of a particular design can only pass signals of a limited frequency range. This places restrictions on the design of electronic component 10, interface circuit 16, and the nature of the signaling schemes used to communicate between them. Further, adding multiple connection paths by electromagnetic coupling is considerably more difficult than adding the extra contacts needed in the case of conductive coupling. Despite these disadvantages, electromagnetic coupling has the particular advantage of providing common-mode electrical isolation between the inductively coupled circuits. This is an important consideration in a medical device, where such isolation is often mandated in patient circuits for safety reasons.

The electrical and pneumatic connections may be arranged independently, literally as depicted in FIG. 2. However, this arrangement is inconvenient for the user. Further, it presents some risk of malfunction if the user neglects to engage both pneumatic and electrical connections. Therefore, in the preferred embodiment, the electrical and pneumatic connections are combined into an integrated hose assembly. Hose 5 and electrical conductors 13 are integrated, such that they are manipulated by the user as a single entity. Further, cuff electrical connector 11 and pneumatic connector 3 are combined, as are the mating electrical connector 12 and pneumatic connector 4, providing simultaneous electrical and pneumatic attachment and detachment. This integration may also be performed at the instrument end of the hose 5 and conductors 13 for pneumatic connector 7 and electrical connector 14. However, in some cases, it may be desirable to maintain independent electrical and pneumatic connections at blood pressure monitoring instrument 8. For example, in certain designs of instrument 8, the pneumatic and electrical connections may lead to widely separated regions of the instrument, making combination of the connections undesirable.

The electrical conductors 13 and hose 5 may be integrated in various ways. In one method, conductors 13 are placed inside the pneumatic lumen of hose 5. In this case, care should be taken that the conductors are small enough in cross section relative to the size of the lumen so as not to obstruct pneumatic flow. According to the design of the connectors used, this construction may present difficulties in achieving leak-free access to the conductors at the terminations of the integrated hose. Therefore, other constructions of integrated hose are suggested in these cases. In one such construction, the hose is furnished with two independent lumens, one of which is used for pneumatic purposes, and the other as a conduit to contain conductors 13. In an alternate construction, conductors 13 are imbedded within the wall of hose 5. For example, the hose may be constructed of extruded thermoplastic, in which case the conductors may be imbedded in the plastic wall during the extrusion process. It is also possible to enclose an ordinary hose and conductors 13 in a common outer jacket, so that they appear as a single integrated entity. In a variation of this method, the conductors may be part of the jacket, as by being imbedded in its wall, or woven into a braided jacket. Hybrid constructions are also possible. For example, one or more of the conductors may be placed within the pneumatic lumen, with the remaining conductors placed in one or more of the other locations described.

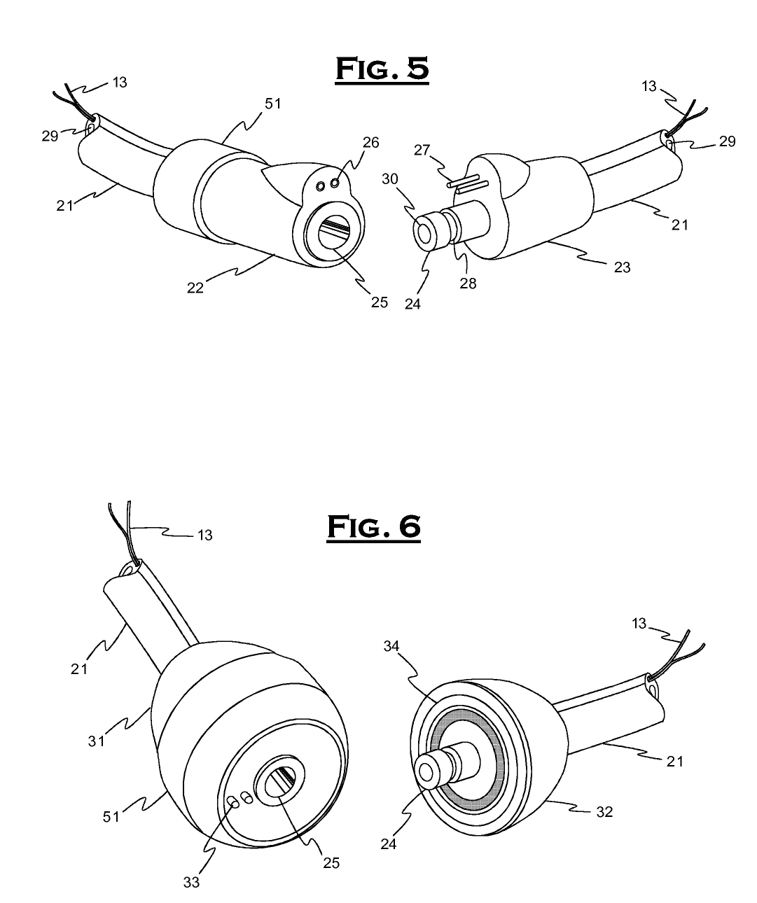

Various forms of the integrated pneumatic and electrical connectors are possible. FIG. 5 shows one embodiment, in which the pneumatic and electrical connectors have been combined side-by-side in common integrated housings. The figure shows an unmated pair, consisting of integrated female connector 22 and integrated male connector 23. Both connectors are shown attached to an integrated hose 21, shown as a broken-away segment, having pneumatic lumen 29 and integrated electrical conductors 13. Male connector 23 is equipped with male pneumatic coupler 24. This pneumatic coupler is furnished with pneumatic passage 30 which communicates with the pneumatic lumen 29 of the attached hose. The male coupler also includes a groove 28 or similar feature designed to engage a locking device to keep the connector mated under tension and pressure. The female connector 22 has female pneumatic socket 25 which communicates with pneumatic lumen 29, and accepts the male coupler 24. The socket is furnished with a locking device, such as a tab, pawl, or ball arrangement, which engages locking feature 28. The locking device may be released, when it is desired to unmate the connectors, by means of sliding collar 51, or similar device such as a release button. The male connector 23 is also equipped with electrical contact pins 27, which are connected to conductors 13 integrated in hose 21. These contact pins engage mating sockets 26 of the female connector 22, which in turn are connected to the integrated conductors 13 of the attached hose. Thus, by inserting male connector 23 into female connector 22, simultaneous electrical and pneumatic connection is secured. By operating the release 51 and separating the male and female connectors, the electrical and pneumatic connections are simultaneously detached. It is understood that other locking means may be employed. For example, sufficient friction may be provided such that an explicit locking device is not required. Or, the locking function could be integrated into the connector body, or electrical contacts, rather than the pneumatic coupler.

In the preferred embodiment, the male pneumatic coupler 24 and the associated socket 25 are made in the form and dimensions of standardized pneumatic connectors, such as the Series 20KA manufactured by Rectus GmbH (Eberdingen-Nussdorf, Germany). In this way, it is possible for conventional blood pressure cuffs, not employing electronic component 10, but using standard connectors, to be mated with socket 25 of female connector 22. In this case, no electrical connection is made to electrical sockets 26. Interface circuit 16 may be designed to detect this condition, and instruct blood pressure monitoring instrument 8 to operate in some fallback mode in which the features permitted by electronic component 10 are not utilized.

Although FIG. 5 shows a particular arrangement of male and female electrical contacts, it is understood that this is not the only possible arrangement. For example, the positions of the male and female contacts may be interchanged, individually or all together. Further, other types of contacts than pins and sockets may be utilized. These include, but are not limited to, butt contacts, bellows contacts, hermaphroditic contacts, coaxial contacts, spring contacts, or any of the other forms well known in the construction of electrical and electronic connectors. Although two electrical contacts and conductors are shown, it is understood that any number may be provided. Further, male pneumatic coupler 24 and socket 25 may be used as an additional electrical contact, or in place of one of the electrical contacts. In this case, pneumatic coupler 24 and pneumatic socket 25 may be of conductive material, or furnished with conductive portions, which touch when mated, and establish electrical contact. The coupler 24 and socket 25, or the conductive portions thereof, are then each connected to one of the conductors 13.

A disadvantage of the arrangement shown in FIG. 5 is that the male connector 23 and female connector 22 must be brought into a certain rotational alignment about their axes before they can be mated. Once mated, relative rotation of the connector male and female parts is precluded. However, an ordinary pneumatic coupler can be mated in any rotational orientation, and once mated can swivel, which is of some benefit in avoiding and remedying tangled hoses. Therefore, it is desirable to preserve this feature in the integrated electrical and pneumatic connector. One configuration which accomplishes this is shown in FIG. 6. In this configuration, the electrical connector pins and sockets have been replaced by a system of sliding annular contacts. In the figure, male connector 32 is provided with concentric annular contact rings 34, which are centered about the axis of male pneumatic coupler 24. The contact rings 34 are connected to the conductors 13, integrated into hose 21 attached to the connector 32. The female connector 31 contains contact points 33, arranged so that they each touch one of the contact rings 34 when the connectors are mated by inserting pneumatic coupler 24 into socket 25. The contact points are connected to conductors 13 integrated in hose 21 attached to connector 31. As this arrangement has symmetry about the axis of the pneumatic coupler 24 and socket 25, it may be mated with any arbitrary rotational alignment. Further, the connectors may be free to rotate relative to each other once mated.

Although the figure indicates that the contact rings 34 are placed on the connector 32 with the male pneumatic coupler 24, and the contact points 33 are placed on the connector 31 with the pneumatic socket 25, it is understood that this placement is arbitrary, and the placement of the rings and contact points may be interchanged. The contact rings 34 may take various forms, such as metal rings imbedded in or attached to the shell of the connector, foil on a printed circuit board, conductive polymer materials, or conductive ink. The contact points 33 may be of various forms, such as spring plungers, leaf spring contacts, elastomeric contacts, dome contacts, or rigid contacts. Although the figure shows only a single contact point per contact ring, it may be desirable to provide multiple contact points per ring, in the interests of providing a redundant contact, or of symmetrically distributing the force of the contact. For example, three contact points located at separations of 120 degrees around the contact ring may be provided.

Although only contact rings and two conductors 13 are shown in the figure, it is understood that any number may be provided. Further, male pneumatic coupler 24 and socket 25 may be used as an additional electrical contact, or in place of one of the electrical contacts. In this case, pneumatic coupler 24 and pneumatic socket 25 should be of conductive material, or be furnished with conductive portions, which touch when mated, and establish electrical contact. The coupler 24 and socket 25, or the conductive portions thereof, are then each connected to one of the conductors 13. A preferred embodiment of this arrangement supports two conductors 13 using the pneumatic coupler and a single concentric annular ring as the contacts.

In an alternate form of the concentric ring contact shown in FIG. 6, the ability of the mated connectors to rotate may be restricted. For example, an alignment key may be provided either to substantially eliminate rotation, or to restrict the rotation to a certain angle, such as less than 180 degrees. In this case, multiple circuits may be accommodated by a single annular contact ring, by dividing the ring radially into sectors, and providing a contact point 33 to mate with each sector. Each sector of the annular ring, and associated contact point, carries the circuit for one of the conductors 13. In this case, the ability of the connectors to rotate when mated may be limited to an angle less than the width of a sector.

Connectors of any of these forms can be made compatible with standard blood pressure connectors, not incorporating electrical contacts, provided that the pneumatic socket 25 or coupler 24 is compatibly dimensioned.

In the contact arrangement of FIG. 6, the force exerted by the contact points 33 against the annular rings 34 is directed axially, and tends to disengage the male and female connector pair. Therefore, to sustain the contact points against the contact rings, the male connector 32 may be securely locked into the female connector 31 when mated. This may be accomplished by means of a locking feature on the male pneumatic coupler 24, or by means of some feature incorporated into the connector shells or bodies, such as tabs, a bayonet lock, a threaded coupling ring, a friction lock, or a detent.

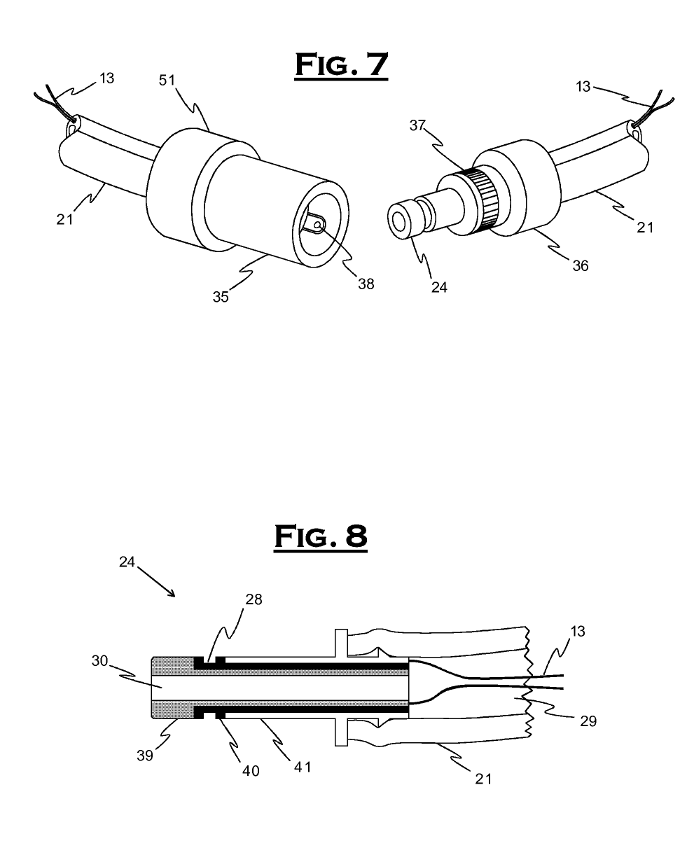

An alternate contact arrangement, in which the contact force does not tend to unmate the connectors, is shown in FIG. 7. In this arrangement, contact forces are directed radially, rather than axially. The figure shows the preferred embodiment, with a contact arrangement supporting two conductors 13, in which one contact is made by means of the pneumatic coupler, and the other by a contact band. The male connector 36 contains pneumatic coupler 24, which is made of a conductive material, or has a conductive portion, and is connected to one of the conductors 13. The male connector also has conductive contact band 37, which is connected to the other conductor. The female connector 35 is has a pneumatic socket (not visible in the perspective) which accepts coupler 24, making pneumatic and electrical connection in the manner already described. The female connector further contains contact finger 38, which touches and establishes electrical contact with band 37 when the connectors are mated. Contact finger 38 and the contact portion of the pneumatic socket are connected to their associated conductors 13.

Because the contact forces are directed radially in the arrangement depicted in FIG. 7, they do not tend to unmate the connectors. Further, by making band 37 of suitable width, this arrangement can be made tolerant of variation in the depth of the connector mating. This arrangement can be held mated with a locking feature on coupler 24, or by means of some feature incorporated into the connector shells or bodies, such as tabs, a bayonet lock, a threaded coupling ring, a friction lock, or detent.

Although the figure shows two conductors and associated contacts, more conductors may be accommodated by adding additional contact bands 37 and contact fingers 38. The contact bands would be arranged parallel to each other, with a contact finger touching each band. Further, although the preferred embodiment utilizes the pneumatic coupler 24 as one of the contacts, this need not be the case if additional contact bands 37 and fingers 38 are provided. Although the figure shows a single contact finger 38 provided per band 37, multiple contact fingers per band may be provided to increase the reliability of the contact, or to distribute the contact force symmetrically.

The connectors shown in FIG. 7 do not require a particular rotational orientation in order to be mated, and are capable of rotating freely once mated. If desired, an alignment key may be added to restrict or eliminate this rotation. In this case, more than one conductor may be supported by a single contact band, by dividing the band into segments, resembling the commutator segments of a DC motor. Each segment would be provided with a contact finger 38, and be connected to one of the conductors 13. The degree of possible rotation of the mated connector may be restricted such that each contact finger remains on its associated contact band segment.

Although FIG. 7 shows the contact ring on the male connector portion 36 and the contact finger 38 on the female portion, it is possible to interchange these components. For example, a contact finger placed on male connector 36 may touch the inside of a contact band provided on female connector 35. In yet a different arrangement, pneumatic coupler 24 may be placed within the female connector body 35, adjacent to contact finger 38, and the pneumatic socket may reside on male connector body 36, concentric with and within contact band 37. Connectors of any of these forms can be made compatible with standard cuff connectors, not incorporating electrical contacts, provided that the pneumatic socket or coupler is compatibly dimensioned, and that sufficient clearance is provided between the pneumatic socket and any surrounding contacts or housing, such that mechanical interference with the standard connector does not result.

In the interests of compactness, the pneumatic coupler and mating socket may be designed to serve as a contact for more than one conductor. FIG. 8 shows a sectional view of a pneumatic coupler 24 providing support for two conductors 13. This arrangement is particularly advantageous if the conductors 13 are to be routed within the pneumatic lumen 29 of hose 21, as it is not necessary to bring the conductors outside the pneumatic lumen to attach them to the contacts. The pneumatic coupler 24 has a central pneumatic passage 30, and is composed of two conductive portions and an insulator 40. One conductive portion 39 is accessible at the tip and inside surface of the coupler. The other conductive portion 41 is accessible at the outer sleeve of the coupler. A locking groove 28 may be provided in the insulator, in one of the conductive portions, or by a gap between the assembled portions. The corresponding female pneumatic socket contains contact areas which individually touch conductive tip 39 and sleeve 41.

In cases where an alignment key is added to restrict free rotation, additional contacts may be provided by dividing one or more of the conductive parts of coupler 24 lengthwise. For example, sleeve 41 could be divided lengthwise to form two independent contact regions on opposite sides of coupler 24. The added contact region so provided could be used in place of, or in addition to, tip contact 39. Although the figure shows two contact regions and conductors, additional contacts and conductors may be added by dividing sleeve 41 into contact bands separated by additional insulators. Further, this pneumatic coupler having two or more circuits may be combined with any of the described connector arrangements where the pneumatic coupler 24 was used as a contact.

The use of electrical contacts may be undesirable under some conditions found in medical practice. For safety reasons, contact between a patient and live electrical circuits should be avoided. As such, patient circuits are often furnished with an isolation barrier, or lacking this, all contacts should be arranged so as to be inaccessible to touch. However, spillage of possibly conductive fluids is a common occurrence in medical care. If such a fluid enters a connector, and reaches the contacts or conductors, electrical leakage to the patient may result. Further, such fluid may cause a malfunction, by causing a shunt path between the contacts. Further, contacts in medical environments are subject to corrosion, damage, and contamination, which may affect their reliability. Therefore, a linkage between electronic component 10 and interface circuit 16 which avoids contacts at least in the region of the patient is highly desirable.

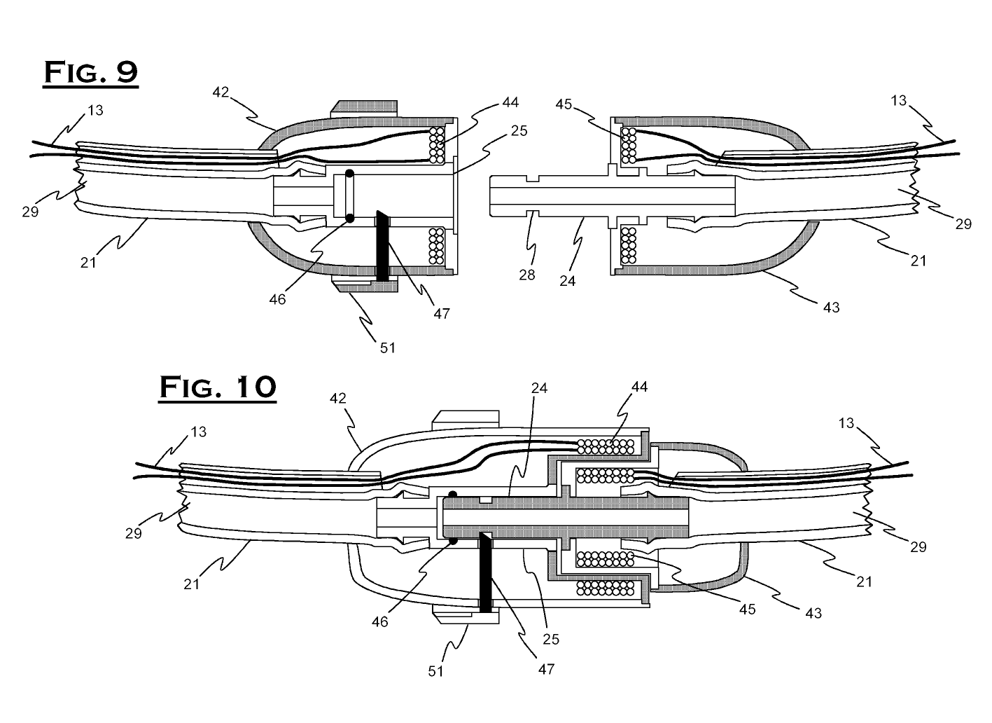

This may be accomplished by inductive coupling. FIG. 9 shows a sectional view of an unmated connector pair employing inductive coupling. Male connector 43 contains the male pneumatic coupler 24 connected to the pneumatic lumen 29 of hose 21. Additionally, it contains coupling coil 45 connected to conductors 13 integrated with hose 21. The coil is wound concentric with coupler 24, and arranged so that the windings lie near the mating face of the connector. The mating female connector 42 has pneumatic socket 25, connected to the pneumatic lumen 29 of hose 21. The socket is equipped with seal 46, which seals against coupler 24 when the coupler is inserted. Locking device 47 engages locking groove 28 of coupler 24 when the connectors are mated. The female connector also contains coupling coil 44, connected to conductors 13 integrated with hose 21. This coil is wound and arranged in a similar fashion to the coil 45 in the male connector. When the connectors are mated, coil 44 becomes positioned adjacent to coil 45, and the coils therefore become at least partially magnetically linked. This results in inductive coupling between the circuits to which the coils are connected.

An alternate arrangement of the inductive coupling coils is illustrated in FIG. 10, which shows a sectional view of a mated connector pair. In this case, rather than the coils being placed adjacent to each other when the connectors are mated, the coils are arranged coaxially, with one coil inside the other. This arrangement permits better coupling of the coils to be obtained.

The coupling of the coils can be improved by the use of magnetic cores or shells surrounding the windings. Such cores of shells may be made from ferrite, iron, nickel alloy, or other such magnetic materials as are commonly used in the cores of transformers or electronic coils. According to the frequencies to be coupled, solid metallic materials may be unsatisfactory, and may require lamination of other well known techniques used to avoid eddy currents and related losses. The magnetic material should be arranged so as to direct the lines of magnetic flux to link both coils. A simple central core will improve coupling. For example, magnetic coupling would be improved in either construction shown in the figures if pneumatic coupler 24 were made of a suitable magnetic material. An outer sleeve of magnetic material, for example placed around the outside of coil 44 of FIG. 10, will have a similar effect. In FIG. 9, each of the coils may be placed in a pot core half, provided with a central hole for the pneumatic coupler 24 and socket 25. The core halves are arranged such that they become assembled into a closed magnetic circuit when the connectors are mated. The best coupling is obtained if the tips of the cores are exposed through the connector housings, such that the two halves of the core may come together with little or no gap when the connectors are mated. However, satisfactory coupling can still be achieved if the core closes with a gap, such as would permit the core tips to lie behind the mating faces of the connectors. By the use of cores or pole pieces, it is not necessary to locate the coils themselves in the mating areas of the connectors. Rather, a core or pole piece may direct the magnetic flux from a coil located elsewhere in the connector housing to the mating area.

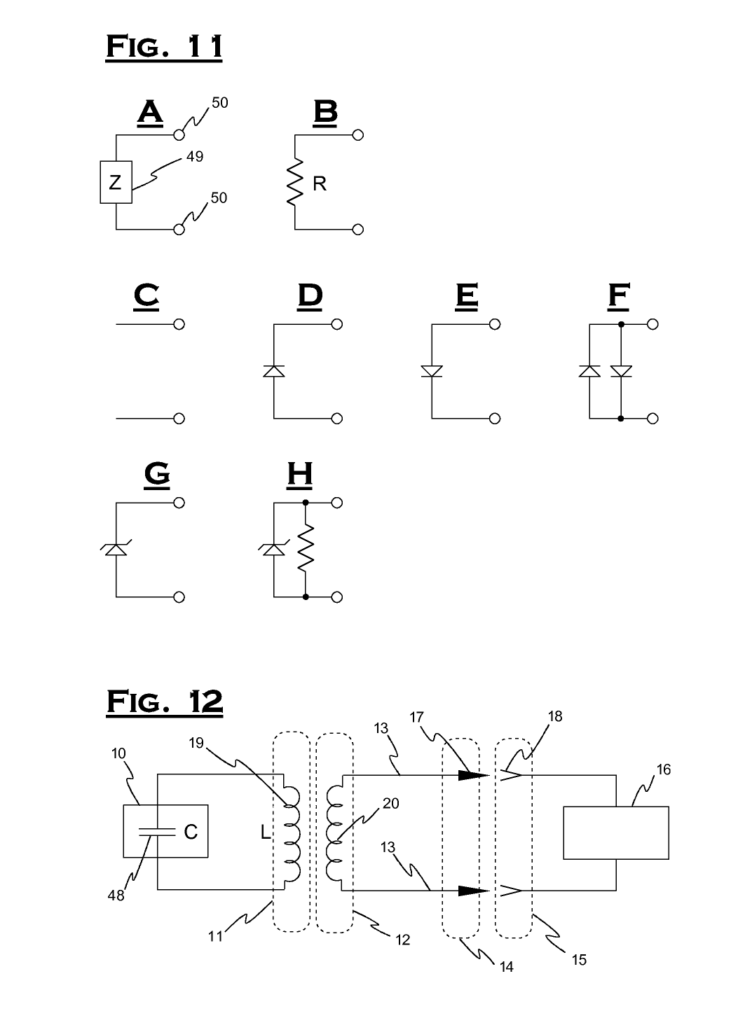

The electronic component 10 attached to the cuff may take a number of forms, according to the purposes for which it is employed and the number of conductors 13 which may be used. In the preferred embodiment, only two conductors 13 are used, to simplify the construction of the connectors. Electronic component 10 is used to identify cuff characteristics. The simplest form of electronic component 10 is a passive network. FIG. 11 shows various simple implementations of cuff identification electronic component 10. The general form is shown in FIG. 11A, in which component 10 comprises a generalized network 49 of impedance Z connected across its terminals 50. The simplest form of network 49 is a resistor, in which case Z equals R, as is shown in FIG. 11B. Various values of the resistance R can be used to represent different values of a cuff property. For example R may be 1000 ohms to denote a neonatal sized cuff, 2000 ohms to denote a pediatric cuff, 3000 ohms to denote an adult cuff, and so on. In theory, a very large number of distinct resistance values are possible, so that a great many cuff types or property values can be encoded, but in practice the number is limited by the tolerance of the resistor and the precision with which it can be measured in light of the effects of the resistance of conductors 13 and the various contacts. A resistor can also be used in the inductively coupled constructions, but in this case there is even greater uncertainty in the measurement of the resistance, due to possible variations in the coupling of the coils. Nevertheless, a useful number of different resistance values, and hence cuff types or property values, can be identified reliably.

In place of a resistor, a capacitor or inductor may be used, with different values of capacitance or inductance representing different cuff types. Networks consisting of combinations of at least two of resistance, capacitance, and inductance may be used. Such networks present a complex impedance Z, with real and imaginary parts. In this case, the real and imaginary parts can encode different aspects of the cuff description. For example, in a network having a series or parallel combination of a resistor and a capacitor, the real component (the resistance) could encode the cuff size, while the imaginary component (the capacitance) may encode some other characteristic, such as reusable vs. disposable.

The impedance Z may also be a non-linear impedance, such as a diode or zener diode. Very simple encoding of a limited number of cuff types or property values is possible in this way. For example, FIG. 11 sections C, D, E, and F show how four types or values may be encoded by simple connection of diodes. In FIG. 11C, the terminals are open circuited, so no current can flow for either polarity of an applied test voltage. In FIG. 11D, a diode is connected across the terminals, such that current will flow only when the lower terminal is positive. In FIG. 11E, the orientation of the diode has been reversed, such that current will flow only when the upper terminal is positive. In FIG. 11F, a pair of antiparallel diodes is used, such that current will flow for both polarities of applied test voltage. Thus, four distinct states can be detected, by simply observing the qualitative presence or absence of current for each polarity of test voltage, without the need for precise measurements. In this case, the antiparallel diodes of FIG. 11F may be replaced by a short circuit across the terminals.

A greater number of states may be detected by more quantitative measurement. In FIG. 11G, a zener diode is connected across the terminals. Zener diodes of different breakdown voltages may be used to encode different values of a cuff property. For example, a breakdown voltage of 5.6 volts could represent a neonatal cuff, 6.8 volts a pediatric cuff, and so on. The polarity of the zener diode may be reversed to double the number of states which may be indicated, or to encode an independent property. For example, the breakdown voltage could encode the cuff size, while the polarity of connection of the diode could represent reusable vs. disposable.

Two independent encoding means may be provided by using a passive component such as a resistor together with a diode or zener diode. FIG. 11H shows a resistor in parallel with a zener diode. The value of the resistor can be measured by applying a small test current or voltage, such that the breakdown voltage of the zener diode is not reached. A large test current can then be used to measure the breakdown voltage of the zener diode without significant influence from the shunting effect of the resistor. The measured value of the resistance and breakdown voltage can be used to independently encode two cuff properties, or combinations of these values can be used to encode a large number of levels of a single property. For example, if four values of resistance, and four values of breakdown voltage are used, these may be combined to encode sixteen levels of some single property. The polarity of connection of the zener diode can also be used as an encoding means. A resistor may also be combined with an ordinary diode, in which case the diode encodes up to three values, according to its polarity of connection (two possibilities) or total absence. Although parallel combinations are simpler to measure, it is evident to those skilled in the art that series combinations are also possible.

A particularly useful construction in the case of inductive coupling is to make electronic component 10 a capacitor. This is shown in FIG. 12, where electronic component 10 consists of a capacitor 48 having capacitance C, which forms a resonant tank circuit with coupling coil 19 having inductance L. The coupling coil itself therefore becomes an element in an L-C impedance network. Different values of resonant frequency, achieved by the use of various values of C or L, can encode different cuff characteristics. For example, a resonance of 100 kHz may denote a neonatal cuff, 150 kHz a pediatric cuff, 200 kHz an adult cuff, and so forth. The resonance of the tank circuit formed by L and C may be determined by interface circuit 16, which is coupled to the tank circuit through coupling coil 20. The measurement of the resonant frequency can be performed by impedance measurement techniques, or interface circuit 16 may consist of an oscillator, the frequency of which is determined by the resonance of the coupled tank circuit. The frequency of this oscillator may be measured by various means, such as by a counter circuit within instrument 8.

The various passive elements described so far encode cuff properties by having their value of impedance, breakdown voltage, or polarity fixed at one of several predetermined values. However, electronic component 10 may have a variable, rather than fixed, characteristic. For example, the resistor in FIG. 11B may be replaced by a thermistor. When this is done, the electronic component serves not to encode cuff properties, but as a sensor, in this case of temperature. In another example, the resistor may be replaced by a piezoresistive sensor, which may be used to sense mechanical force, such as that resulting from the pressure or pulse signal. Similarly, sensors which vary in capacitance or inductance may be used. A sensor may also be connected in combination with a fixed identifier. For example, a thermistor may be connected in shunt with a zener diode, such that the thermistor resistance indicates the variable temperature, while the zener diode breakdown voltage indicates some fixed property, such as cuff size.

Electronic component 10 may also contain active, rather than just passive, electronic elements. For example, electronic component 10 may be an electret microphone cartridge. An electret microphone cartridge, which may be used to acquire the pulse signal, consists of a transducer and a preamplifier in a single package with two terminals, which serve to both power the device and carry the signal.

However, besides analog transducers, certain digital devices use only two terminals to both power the device and carry information. Examples are found in the “One-Wire” protocol devices manufactured by Dallas Semiconductor (Dallas, Tex.). These devices parasitically derive power from a single bi-directional digital signaling line, which utilizes a serial protocol to exchange data with the device. Some of these devices are eminently suited for encoding cuff properties by a digital code. For example, the Dallas Semiconductor DS2401 device digitally encodes a customizable 48 bit number. Some of these bits can be used to encode descriptors of cuff characteristics, such as size or type. If desired, the remaining bits can be used as a unique individual cuff serial number or individual identifier. Such a cuff serial number can also be used as a patient identifier, in which case the cuff, particularly a disposable one, become an identification armband, taking the place of the wristband commonly used for patient identification.

The DS2401 is effectively a read-only memory device, the programming of the 48 bit number being possible only during manufacture of the device. A device including non-volatile memory that can be written as well as read allows additional functionality. For example, the number of uses of the blood pressure cuff can be recorded, so that the user can be advised when the cuff is worn out and replacement is necessary. If the cuff is assigned to a particular patient, as when it is used as a patient identifier arm band, patient data may be recorded in cuff electronic component 10. Examples of suitable readable and writable devices include the Dallas Semiconductor DS2300A and related devices, containing EEPROM memory, which while non-volatile, may be erased and rewritten at any time.

It is also possible to include sensor data, in addition to stored digital data, in the information communicated by electronic component 10. For example, the Dallas Semiconductor DS1820 family of devices contain a temperature sensor, and are capable of communicating a digital representation of the temperature in addition to a numeric identifier, while using only two terminals for both power and data exchange. A blood pressure cuff including such a device could not only provide a numeric identifier encoding the cuff properties, but also report the temperature of the person or animal to which it is attached. It is obvious to those skilled in the art that the same techniques used to acquire and condition the temperature sensor signal could be applied to other types of sensors.

Digital devices, such as the Dallas Semiconductor devices described above, are well suited for use as electronic component 10 in cases where a conductive connection to interface circuit 16 is used, as is shown in FIG. 3. However, devices of this type can be used only with great difficulty in inductively coupled arrangements, such as that shown in FIG. 4. This is because the signaling scheme used to power and communicate with these devices requires transfer of energy down to very low frequencies, preferably including DC. This is problematic for an inductive coupling scheme, as it requires that the coils have very great inductance, which is inconvenient. Further, the signaling scheme also requires the transmission of high frequencies, requiring that these same coupling coils be designed to pass a great range of frequencies. Such coils are objectionable on practical grounds, as their construction is difficult if even feasible.

These objections can be overcome by a signaling scheme which carries the power and intelligence on a radio frequency (RF) carrier. In this case, the coils need be designed to operate only in a narrow band surrounding a particular carrier frequency, which may be selected with convenience of construction of the coils in mind. In the arrangement shown in FIG. 4, interface circuit 16 would generate an RF carrier, which would be passed from coil 20 to coil 19 by inductive coupling. Electronic component 10 would receive this RF energy, and rectify it to serve its power requirements. This same RF carrier which is rectified to provide power can also be modulated with information, allowing interface circuit 16 to send information to electronic component 10. Electronic component 10 is able to communicate back to interface circuit 16 in various ways. For example, electronic component 10 may apply a variable loading to coil 19, the effects of which, reflected to coil 20, may be sensed by interface circuit 16. This scheme, sometimes known as absorption modulation, may transmit the logic states corresponding to a digital serial data stream. Alternately, electronic component 10 may produce an output at a second carrier frequency, which is carried through the coupled coils to interface circuit 16. This second carrier frequency may be modulated with information to be sent from electronic component 10 to interface circuit 16.

Devices operating on these principles are well known in commerce, and inexpensively mass produced. In a field known as radio frequency identification (RFID) similar principles are used to allow an interrogation device, often called a reader, to read information stored in a tag or identification card, which may be attached to an object or person. The tag contains an integrated circuit known as an RFID transponder, which is connected to a small coupling coil. The reader contains a coupling coil, connected to suitable electronics. In operation, the coil of the reader is brought near the coil of the tag, and power and data are exchanged as described above. The tag may be considered equivalent to electronic component 10 and coil 19 of FIG. 4, while the reader is equivalent to interface circuit 16 and coil 20. A particular feature of RFID devices is that they are designed to work with very loose coupling between the transponder and interrogator coils. As such, if commercial RFID devices are used to implement the system of FIG. 4, there is great freedom in design of the coils 19 and 20, and the connectors 11 and 12 in which they reside, as obtaining tight coupling is no longer a design priority. Various manufactures produce RFID devices operating on similar principles. As an example, the HT2MOA2S20 transponder manufactured by Phillips Semiconductors (Eindhoven, The Netherlands) is suitable for use as electronic component 10. This device includes a unique identifier number, plus a user programmable EEPROM memory. A corresponding reader device is the Phillips Semiconductor type HTCM400, which is suitable for use as interface circuit 16. These devices operate at a carrier frequency of 125 kHz, which is well adapted to convenient construction of the coupling coils. It is obvious to those skilled in the art that the same techniques used to construct these commercial transponders can be used to create transponders which accept and condition sensor inputs for transmission to interface circuit 16. Although FIG. 4 shows a single use of inductive coupling at one end of conductors 13, it is understood that inductive coupling may be employed at either or both ends of the conductors.

In FIG. 2, electronic component 10 is illustrated as being directly attached to cuff 1. However, in many cases, electronic component 10 is more conveniently placed within the housing of cuff connector, in proximity to the contacts or coupling coil. When the pneumatic and electrical connections are combined into a single connector body as has been described, and electronic component so located remains associated with the cuff, since the connector body containing the component is attached to the cuff by the short tube 2. In cases where electronic component 10 includes a sensor, the sensor may need to be mounted on the cuff itself, as in the case of a temperature sensor intended to measure the temperature of the limb encircled by the cuff. In such a case, the remaining elements of electronic component 10 may be located either on the cuff or within the body of the connector.

It is of course possible to use more than two conductors 13, and a suitable number of contacts or electromagnetic coupling links to support them. If this is done, the electronic component 10 may take alternate forms which use these additional conductors to advantage. For example, separate conductors can be used to supply power and exchange signals. Multiple conductors may be used to exchange the data, such as a clock signal in addition to the data signal. Separate conductors may be used to encode different cuff properties. For example, if three conductors are used, one may be designated as common, a resistor connected from the second to the common may encode the cuff size, while the resistance between the third and common may represent some other cuff property. These and similar variations are contemplated as being part of the disclosure.

What is claimed is:

1. A blood pressure measurement device comprising:
a blood pressure cuff detachably connected to a blood pressure monitoring instrument by means of a hose assembly and connectors, in which the blood pressure cuff contains an electronic component capable of exchanging information with the blood pressure monitoring instrument, the hose assembly is provided with electrical conductors in addition to one or more pneumatic lumens, and at least the mating connectors between the cuff and hose assembly are provided with electrical coupling in addition to pneumatic coupling, such that simultaneous electrical and pneumatic connection is established between the cuff and instrument when the cuff is connected to the hose.

2. The device of claim 1, in which the electronic component includes encoding of properties of the cuff.

3. The device of claim 1, in which the electronic component includes a sensor.

4. The device of claim 1, in which the electronic component is an impedance network.

5. The device of claim 4, in which the impedance network is a resistor.

6. The device of claim 4, in which the impedance network contains non-linear elements.

7. The device of claim 1, in which the electronic component contains a read-only memory device.

8. The device of claim 1, in which the electronic component contains a re-writable memory device.

9. The device of claim 8, in which the memory device is used to store the number of uses of the cuff.

10. The device of claim 8, in which the memory device is used to store patient data.

11. The device of claim 1, in which the blood pressure cuff electronic component includes encoding of a unique identifier or serial number.

12. The device of claim 11, in which the blood pressure cuff and its associated unique identifier are used as a patient identifier.

13. A blood pressure hose assembly having integrated electrical conductors in addition to one or more pneumatic lumens.

14. The blood pressure hose assembly of claim 13, in which the electrical conductors are located within a pneumatic lumen of the hose.

15. The blood pressure hose assembly of claim 13, in which the electrical conductors are located in a lumen not used for pneumatic purposes.

16. The blood pressure hose assembly of claim 13, in which the electrical conductors are imbedded in the wall of the hose.

17. The blood pressure hose assembly of claim 13, in which a common outer jacket integrates the electrical conductors with the pneumatic portion of the hose.

18. A blood pressure cuff connector integrating electrical connection as well as pneumatic connection, such that the pneumatic and electrical circuits are simultaneously engaged when the connector is mated.

19. The connector of claim 18, in which the electrical connection is made by one or more electrical contacts located adjacent to the pneumatic connection.

20. The connector of claim 18, in which one or more electrical contacts are arranged as annular rings surrounding the pneumatic connection.

21. The connector of claim 18, in which one or more electrical contacts are arranged as a band surrounding the pneumatic connection.

22. The connector of claim 18, in which the pneumatic connection fitting also serves as an electrical contact.

23. The connector of claim 22, in which the pneumatic connector is divided by insulation material, such that it carries more than one electrical connection.

24. The connector of claim 18, in which the electrical connection is made by means of inductive coupling coils surrounding the pneumatic connection.

25. The connector of claim 24, in which the inductance of the coupling coil forms part of an impedance network.

26. A blood pressure measurement method comprising:
providing a blood pressure cuff detachably connectable to a blood pressure monitoring instrument by means of a hose assembly and connectors;
providing the blood pressure cuff with an electronic component capable of exchanging information with the blood pressure monitoring instrument;
providing the hose assembly with electrical conductors in addition to one or more pneumatic lumens; and
providing at least the connectors between the cuff and hose assembly with electrical coupling in addition to pneumatic coupling, such that simultaneous electrical and pneumatic connection is established between the cuff and instrument when the cuff is connected to the hose.
